(19) United States
(12) Patent Application Publication
(10) Pub. No.: US 2004/0162613 A1
(43) Pub. Date: Aug. 19, 2004

(54) Cosmetic and reconstructive prosthesis containing a biologically compatible rupture indicator

(76) Inventor: Thomas Roballey, Huntington, CT (US)

Correspondence Address: MELVIN K. SILVERMAN, 500 WEST CYPRESS CREEK ROAD, SUITE 500, FT. LAUDERDALE, FL, 33309, US

(21) Appl. No.: 10/773,604

(22) Filed: Feb. 5, 2004

Related U.S. Application Data
(60) Provisional application No. 60/445,227, filed on Feb. 6, 2003.

(51) Int. Cl. A61F 2/12; A61F 2/04

(52) U.S. Cl. 623/8; 623/23.67

(57) ABSTRACT

A cosmetic and reconstructive prosthesis containing a rupture indicator is provided, which includes an external envelope of medical grade elastomer containing a fluid material and a biologically compatible chemical indicator for indicating rupture of the prosthesis, and an internal envelope of medical grade elastomer disposed within the external envelope, the internal envelope containing an implant filling material. Also provided is a method of detecting rupture of a prosthesis, which includes surgically implanting a prosthesis containing a biologically compatible chemical indicator in a location of a patient body in need of the prosthesis; and detecting a change of a body secretion or peripheral blood, or a change locally around the prosthesis for indication of leaking out of the indicator from the prosthesis.

### CROSS REFERENCE TO RELATED APPLICATIONS

This application claims the benefit under 35 USC 119 (e) of the provisional patent application Serial No. 60/445,227, filed on Feb. 6, 2003, which is herein incorporated by reference in its entirety.

### FIELD OF THE INVENTION

The present invention relates in general to the field of cosmetic and reconstructive prosthesis, and more particularly to a prosthesis, such as breast prosthesis, containing a biologically compatible chemical indicator for indicating rupture of the prosthesis.

### BACKGROUND OF THE INVENTION

Almost any part of the body can be filled to create balance and harmony. Often by adding to an area, it can affect the whole face or body. Today implants are widely used in cosmetic and reconstructive corrections. One of the most commonly used substances as the filling material is silicone. It has been used for various facial implants, such as brow, nose, cheek, chin and lips, and various body implants, such as pectoral and breast, triceps and biceps, genitals, buttocks and calf. Among all types of cosmetic and reconstructive implants, breast implant has the largest number of implementation, hence is addressed with specific emphasis hereinafter.

Over the last three decades, surgical breast augmentation in the United States has been primarily done by placement of breast implants. Implants are surgically placed either in front of the pectoralis major muscle—called subglandular or pre-pectoral implants—or they are placed behind the pectoralis major muscle called submuscular, retroglandular or retropectoral implants.

The type of the material in the implants and the variations in the shape and supporting shells in the implant also categorize implants. A silicone implant is composed of silicone gel contained within a silicone polymer membrane or envelope. A saline implant refers to an implant composed of saline within a silicone polymer membrane. A double-lumen implant refers to an implant having two shells, typically an inner shell filled with silicone surrounded by an outer shell filled with saline. A reverse double-lumen implant refers to an inner shell of saline surrounded by silicone. Other variations have been implanted with three or more shells. The term “stacked” implants refers to cases when multiple implants are placed on top of each other in the same breast to achieve larger volumes.

Before 1992, the majority of breast augmentation implants in the United States contained silicone gel. This was due to general acceptance by the medical community at the time, surgeons' preference, and the reported better texture and “feel” of a silicone implant versus a saline implant by the patients. It has been estimated that over one million women in the United States alone have received silicone breast implants.

In the 1980s, independent authors questioned a possible association between silicone implants and the subsequent development of connective-tissue diseases. Fueled by media hype and class action lawsuits, the Food and Drug Administration (FDA) was asked to analyze the data and make a decision. In 1992, the FDA announced that breast implants containing silicone gel would only be available in the United States under clinically controlled trials. It has since been primarily restricted in the United States to women undergoing post-mastectomy reconstruction and those requiring secondary surgery after breast augmentation. Saline breast implants have replaced silicone implants as the common breast prosthesis in the past decade. However, in comparison to silicone gel implants, saline breast implants are inferior in terms of mimicking elasticity, feel, movement of the natural breast tissue.

Since 1992, there are many studies investigating the safety concerns of the silicone gel implants. In 1999, after reviewing dozens of studies, the Institute of Medicine (IOM) concluded in its landmark 1999 report that silicone gel implants do not cause the autoimmune disorders such as lupus or arthritis. The main safety concern according to the report is the implants' tendency to rupture. The silicone can bleed or leak out of its shell, causing infections. The IOM 1999 report became the turning point for the breast implant industry and clinics, opening the door for returns of silicone breast implants for cosmetic use.

Silicone implant rupture is often locally symptomatic, however, continues to be a genuine clinical concern for patients and physicians. In the United States, an estimated one to two million patients, or approximately 1% of the adult female population, have breast implants. The risk of silicone rupture increases with the age of the implant. One recent study revealed that the median lifespan of a silicone gel breast implant is 16.4 years. In that study, 79.1% of implant were intact at 10 years; the percentage decreased to 48.7% at 15 years.

Another study revealed that at least 77% of 344 women from Birmingham, Ala. who were not referred for examination had at least one implant that “ruptured” or had an “indeterminate” finding at MR imaging. The reported median implant age at rupture was 10.8 years, and submuscular implants were more likely than subglandular implants to rupture.

In essentially all patients, a fibrous capsule forms around the implant (ie, encapsulation). The capsule may be soft and nonpalpable or hard and resistant. Two types of silicone gel breast implant rupture can occur: intracapsular rupture occurs when silicone escapes the elastic membrane shell but is contained in the fibrous capsule. This form of silicone gel breast implant rupture is most common. Extracapsular rupture involves the escape of free silicone gel through the fibrous capsule, with extravasation into the breast tissue. Migration of silicone to the axillary lymph nodes also may be present. Furthermore, silicone gel can migrate to the brachial plexus, chest wall, axilla and the wrist.

The diagnosis of silicone gel breast implants rupture is useful to both clinicians and patients; it aids in surgical decision-making and helps the patient gain peace of mind. Furthermore, the systemic effects of leaked silicone breast implants, if any, remain unclear. Currently, magnetic resonance imaging (MRI) is used to reliably evaluate silicone gel breast implants, because the findings at clinical examination often are nonspecific. However, MRI is an expensive examination involving complex instrument and data processing.

The above-described problems also present with other cosmetic and reconstructive implants using silicone as the implant filling material, such as brow, nose, cheek, chin, lips, pectoral, breast, triceps and biceps, genitals, buttocks and calf. Among these, some require a small amount of implant filling material, some require a large amount of filling material. For example, in the case of calf, the calf implant is inserted to rebalance legs affected by such diseases as polio, which requires relatively large amount of filling material. In general, the larger the amount of implant filling material is, the worse the potential impact of filling material to a patient can be. Therefore, it is apparent there exists a need for cost effective and more convenient test methods for detection of the rupture of the silicone breast implants and other silicone cosmetic and reconstructive implants.

On the other hand, various biocompatible dyes have been used in pharmaceuticals or food industries for human use. For example, U.S. Pat. No. 6,020,374 teaches various synthetic dye compounds for pharmaceutical uses, such as Aurintricarboxylic acid (ATA), Halogenated ATA, Sulfonated ATA, Sulfonated-Halogenated ATA, Phosphorylated ATA, Anazolene Sodium, Eosine I Bluish, Eosine Yellowish, Erythrosine, Evan's Blue (EB), Fast Green FCF, Fuchin(e) Acid, Iodophthalein Sodium, Rose Bengal, Sulfobromophthalein Sodium, Suramin Sodium, Trypan Blue, Trypan Red, Rosaniline Chloride, Crystal Violet, Methyl Blue, Methyl Green, Coomassie Blue, Basic Fuchsin, Malachite Green, Brilliant Green, Aniline blue, Brilliant Cresyl Blue, Safranin O, Ethyl Violet, Pararosaniline Acetate, Methyl Violet, Direct Yellow, Direct Red, Ponceau S, Ponceau SS, Nitrosulfonazo III, Chicago Sky Blue 6B, and Calcion or RG-13577.

However, the biocompatible dyes have not been utilized for indicating or detection of rupture of breast implants.

### SUMMARY OF THE INVENTION

In one embodiment, the present invention is directed to a cosmetic and reconstructive prosthesis containing a rupture indicator, which comprises an external envelope of medical grade elastomer containing a fluid material and a biologically compatible chemical indicator for indicating rupture of the breast prosthesis, and an internal envelope of medical grade elastomer disposed within the external envelope, the internal envelope containing an implant filling material.

The biologically compatible chemical indicator can be a dye, such as methylene blue and various other dyes described in detail in the specification; an odour generating agent which generates a non-human body smell when leaking out from the prosthesis; a sensation agent which causes a local sensation when leaking out from the prosthesis.

The cosmetic and reconstructive prosthesis containing a rupture indicator includes breast, brow, nose, cheek, chin, lips, pectoral, triceps and biceps, genitals, buttocks and calf prosthesis.

In a further embodiment, the present invention is directed to a method of detecting rupture of a cosmetic and reconstructive prosthesis. The method comprises surgically implanting a prosthesis containing a biologically compatible chemical indicator for indicating rupture of the prosthesis in a location of a patient body in need of the prosthesis; and detecting a change of a body secretion or peripheral blood for indication of leaking out of the indicator from the prosthesis. The body secretion that can be used for the detection includes urine, saliva, perspiration and feces. The change includes a presence of the chemical indicator or metabolized product thereof in the body secretion or peripheral blood, an odour from the indicator in the body secretion, and a color change of at least one of the body secretion.

In another embodiment, method of detecting rupture of a cosmetic and reconstructive prosthesis comprises surgically implanting a prosthesis containing a biologically compatible chemical indicator for indicating rupture of the prosthesis in a location of a patient body in need of the prosthesis; and detecting a change locally around the prosthesis for indication of leaking out of the indicator from the prosthesis. The change includes a local skin color change, a local sensation, and a local x-ray opacity change from that after the surgically implanting the prosthesis.

### BRIEF DESCRIPTION OF THE DRAWINGS

The above and other objects, features and other advantages of the present invention will be more clearly understood from the following detailed description taken in conjunction with the accompanying drawings, in which:

FIG. 1 is a side view of the double lumen breast prosthesis with the external envelope containing a chemical indicator of one embodiment of the present invention.

FIG. 2 is a side view of the double lumen breast prosthesis with the external lumen containing a chemical indicator and a filling tube of one embodiment of the present invention.

### DETAILED DESCRIPTION OF THE INVENTION

In one embodiment, the present invention provides a breast prosthesis containing a rupture indicator comprising an external envelope of medical grade elastomer containing a fluid material and a biologically compatible chemical indicator for indicating rupture of the prosthesis, and an internal envelope of medical grade elastomer disposed within the external envelope, the internal envelope containing an implant filling material.

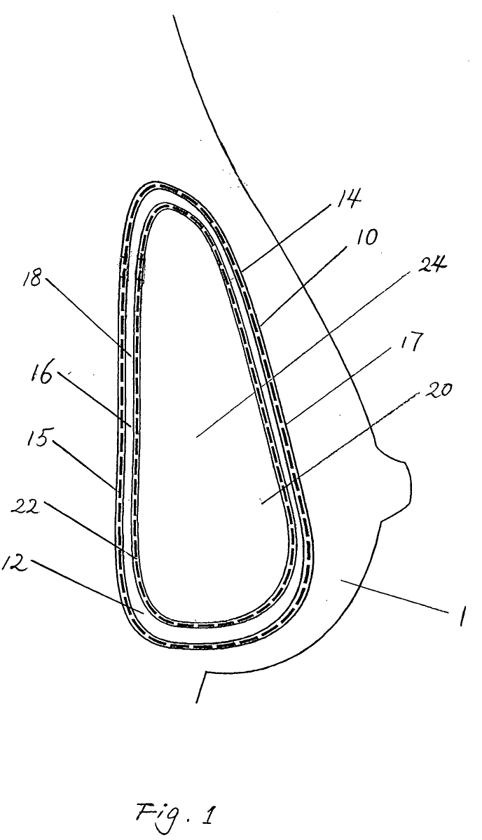

As shown in FIG. 1, a breast prosthesis 10 implanted in a human breast 1 includes an external lumen 12 defined by an external envelope 14. The external lumen 12 is filled with a fluid material 16 containing a chemical indicator 18. Preferably the fluid material has a low viscosity such as a saline solution. The breast prosthesis 10 also includes an internal lumen 20 defined by an internal envelope 22. The internal lumen 20 is filled with an implant filling material 24, preferably much higher viscosity such as silicone gel. Suitable examples of implant filling materials include, but not limited to, glycosaminoglycan including hyaluronic acid, chondroitin 4-sulfate, chondroitin 6-sulfate, dermatan sulfate, heparin sulfate, and keratan sulphate; mucopolysaccharide, polyvinylpyrollidone, polyvinyl pyrralidone, polyvinyl alcohol, polyacrlimides, polysaccharides, hydroxypropylmethyl cellulose, polyethylene oxide, hyaluronic acid, sodium or calcium alginate, hydrogel polyurethane, hydroxyethyl starch, polyglycolic acid, polyacrylamide, hydroxyethylmethacrylate (HEMA), and naturally derived biopolymers including sodium kinate, seaweed, and agar; aqueous solution of polyethylene glycol; linear or branched, or cross-linked polyacrylamide, sodium hyaluronate, phosphatidylcholine (PC), hydroxypropylmethyl cellulose (HPMC) and its derivatives including hydroxyalkyl cellulose, sodium carboxymethyl cellulose, hydroxyethyl cellulose, hydroxypropyl cellulose, methylhydroxypropyl cellulose, methyl cellulose and ethylhydroxyethyl cellulose; and polyoxyethylene/polyoxypropylene block copolymers which have gelling properties at body temperature. Furthermore, the implant filling material can also be a saline solution.

The external envelope 14 and internal envelope 22 may be made of various soft flexible biocompatible materials such as a silicone elastomer. Preferred materials include silicone elastomers such as polydimethylsiloxane or polymethylvinylsiloxane or copolymers thereof with other substances. Other polymers may be substituted as will be apparent to those skilled in the art.

As illustrated, the external envelope 14 has a generally tear-drop shape with a relatively flat rear portion 15 and rounded dome or a forward surface 17. The external envelope 14 defines an external lumen which may be of a generally tear-drop shape or other non-symmetrical shape in order to conform to the contours of a human breast. It should be recognized that in certain cases a round shape may be needed.

The biologically compatible chemical indicator can be several types. One type of chemical indicators is biocompatible dyes. Suitable examples include, but not limited to, aurintricarboxylic acid (ATA), halogenated ATA, sulfonated ATA, sulfonated-halogenated ATA, phosphorylated ATA, anazolene sodium, eosine I bluish, eosine yellowish, erythrosine, Evan's blue (EB), fast green FCF, fuchin(e) acid, iodophthalein sodium, rose bengal, sulfobromophthalein sodium, suramin sodium, trypan blue, trypan red, rosaniline chloride, crystal violet, methyl blue, methyl green, methylene blue, coomassie blue, basic fuchsin, malachite green, brilliant green, aniline blue, brilliant cresyl blue, safranin O, ethyl violet, pararosaniline acetate, methyl violet, direct yellow, direct red, ponceau S, ponceau SS, nitrosulfonazo III, chicago sky blue 6B, calcion or RG-13577, and commonly used food dyes such as FD&C red No. 3, FD&C red No. 40, FD&C blue No. 1 and FD&C yellow No. 5. In a preferred embodiment, methylene blue is used.

Preferably, the dye is water soluble so that it can release out through body secretion, such as urine, saliva, perspiration, and feces, or in peripheral blood when the prosthesis ruptures. When the prosthesis ruptures, even a minor rupture, chemical indicator 18 leaks out from external lumen 12 to the human body. Optionally, chemical indicator 18 can also be contained in the internal lumen 20, which will leak out when both envelopes rupture. In one embodiment, the leaked chemical indicator 18 can be visually detected in urine, or saliva. It can also be detected in a body secretion sample or a peripheral blood sample using a colorimetric method. Such detection can be performed in a clinical laboratory, or can be performed using a specifically designed kit for home use, similar to the glucose, or pregnancy test kits. The Example described hereinafter provides a detailed configuration of the breast implant of the present invention and the method of detection. In the case of breast implant, the filling material in the internal lumen is 85% or more of the total volume of the prosthesis for maintaining the overall prosthesis properties, and the fluid material in the external lumen is 15% or less. The ratio between the filling material and the fluid material in the external lumen can be different for different types of prosthesis.

With water soluble dyes, the rupture can also be detected by staining of skin locally by the leaked dye. Furthermore, in addition to dyes, other non-coloring biocompatible chemical indicators, detectable at a trace amount, can also be used, which can be detected in body secretion, such as urine, saliva, perspiration and feces, or in peripheral blood, using a chemical reaction which is sensitive and specific to the indicator.

Another type of the biologically compatible chemical indicators is an odour generating material which causes a smell change of body secretion, such as saliva, urine, perspiration and feces. One example is a sterilized garlic solution. When the breast prosthesis ruptures, the odour generating solution leaks into body, which can cause an unusual body odour, hence, alert the user.

A further type of the biologically compatible chemical indicators is a sensation agent which causes a sensation, such as local pain, burning, or irritation. One example is a very dilute capsaicin solution, which can cause a local burning sensation when leaking out.

Moreover, a further type of chemical indicator is a material which causes temporary local tissue x-ray opaque. Using this type indicator, a simple mammogram at annual routine examination of a user can detect the leak from the rupture.

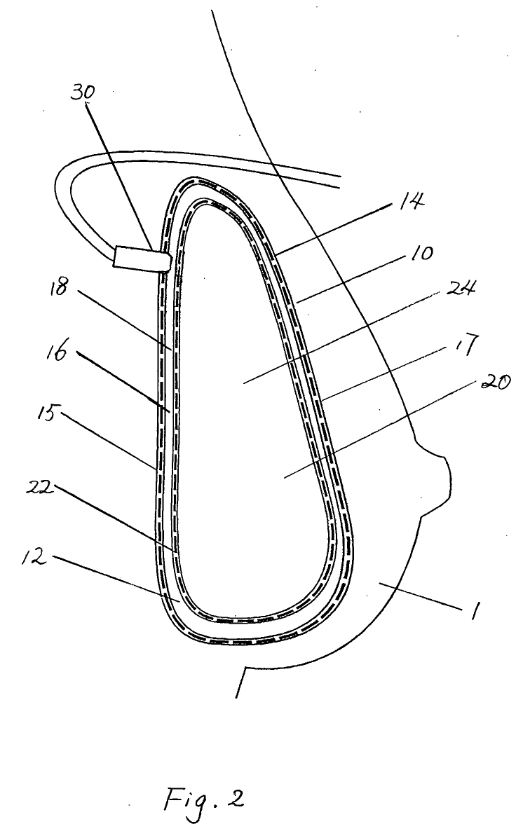

A further embodiment of the present invention includes means for adding or removing first fluid material 16 to or from the external lumen 12 and/or second material to or from internal lumen 20. One such means is illustrated in FIG. 2. As shown, a filling tube 30 is in an inserted position within the external lumen 12 and can be inserted at the time of manufacture. Alternatively, a filling tube can be inserted later. The filling tube 30 is typically inserted through a self-sealing valve (not shown) commonly used in breast implant surgery. Preferably, a relatively soft material is used for filling tube 30 so as not to puncture the envelopes. The distal end of filling tube 30 is connected with a source of the second material, such as a saline solution (not shown). Upon completion of the filling process, the filling tube 30 is removed and the self-sealing valve closes.

Using the breast prosthesis containing a rupture indicator and the method of detection, the potential rupture of the breast prosthesis can be conveniently detected. With the present invention, an early detection of the rupture is possible. Since when chemical indicator contained in the external lumen 12 leaks out, it indicates a potential problem of the breast prosthesis, even if the internal envelope has not ruptured. A further confirmation examination can be performed using MRI.

### EXAMPLE

A double lumen breast implant having a structure shown in FIG. 1 has silicone gel commonly used in the breast implant as the filling material inside the internal lumen 20. The external lumen contains from about 35 to about 45 ml of sterilized aqueous solution of methylene blue. The methylene blue is in a concentration range from about 1 mg/ml to about 4 mg/ml. With the concentration and volume of the methylene blue described, it is in a range from about 1 to about 2 mg per kilogram of body weight for an average female (from about 50 to about 70 kg). In the event of rupture, the methylene blue solution leaks out from the external lumen, metabolizes in kidney, and releases to urine, which causes a color change of the urine.

The biocompatible chemical indicators and the method of detection of implant rupture are specifically described using breast prosthesis. It should be understood, however, the materials and the methods are can also be used for other cosmetic and reconstructive prostheses, such as brow, nose, cheek, chin, lips, pectoral, triceps and biceps, genitals, buttocks and calf.

While the present invention has been described in detail and pictorially shown in the accompanying drawings, these should not be construed as limitations on the scope of the present invention, but rather as an exemplification of preferred embodiments thereof. It will be apparent, however, that various modifications and changes can be made within the spirit and the scope of this invention as described in the above specification and defined in the appended claims and their legal equivalents.

What is claimed is:

1. A prosthesis containing a rupture indicator comprising:
(a) an external envelope of medical grade elastomer containing a fluid material and a biologically compatible chemical indicator for indicating rupture of said prosthesis, and
(b) an internal envelope of medical grade elastomer disposed within said external envelope, said internal envelope containing an implant filling material.

2. The prosthesis containing a rupture indicator of claim 1, wherein said biologically compatible chemical indicator is a dye.

3. The prosthesis containing a rupture indicator of claim 2, wherein said biologically compatible chemical indicator is methylene blue.

4. The prosthesis containing a rupture indicator of claim 2, wherein said biologically compatible chemical indicator is at least one selected from the group consisting of aurintricarboxylic acid (ATA), halogenated ATA, sulfonated ATA, sulfonated-halogenated ATA, phosphorylated ATA, anazolene sodium, eosine I bluish, eosine yellowish, erythrosine, Evan's blue (EB), fast green FCF, fuchin(e) acid, iodophthalein sodium, rose bengal, sulfobromophthalein sodium, suramin sodium, trypan blue, trypan red, rosaniline chloride, crystal violet, methyl blue, methyl green, coomassie blue, basic fuchsin, malachite green, brilliant green, aniline blue, brilliant cresyl blue, safranin O, ethyl violet, pararosaniline acetate, methyl violet, direct yellow, direct red, ponceau S, ponceau SS, nitrosulfonazo III, chicago sky blue 6B, calcion or RG-13577, FD&C red No. 3, FD&C red No. 40, FD&C blue No. 1 and FD&C yellow No. 5.

5. The prosthesis containing a rupture indicator of claim 1, wherein said biological compatible chemical indicator is an odour generating agent which generates a smell when leaking out from said prosthesis.

6. The prosthesis containing a rupture indicator of claim 1, wherein said biological compatible chemical indicator is a sensation agent which causes a local sensation when leaking out from said prosthesis.

7. The prosthesis containing a rupture indicator of claim 1, wherein said prosthesis is a breast prosthesis.

8. The prosthesis containing a rupture indicator of claim 1, wherein said prosthesis is at least one selected from the group consisting of brow, nose, cheek, chin, lips, pectoral, triceps and biceps, genitals, buttocks and calf prostheses.

9. The prosthesis containing a rupture indicator of claim 1, wherein said external lumen further comprises a filling means for filling said fluid material.

10. The cosmetic and reconstructive prosthesis containing a rupture indicator of claim 9, wherein said filling means is a self-sealing valve.

11. A method of detecting rupture of a cosmetic and reconstructive prosthesis comprising:
(a) surgically implanting a prosthesis containing a biologically compatible chemical indicator for indicating rupture of said prosthesis in a location of a patient body in need of said prosthesis; and
(b) detecting a change of a body secretion or peripheral blood for indication of leaking out of said indicator from said prosthesis.

12. The method of claim 11, wherein said body secretion is at least one selected from the group consisting of urine, saliva, perspiration and feces.

13. The method of claim 12, wherein said change is a presence of said chemical indicator or metabolized product thereof in said body secretion or peripheral blood.

14. The method of claim 12, wherein said change is an odour from said indicator in said body secretion.

15. The method of claim 12, wherein said change is a color change of at least one of said body secretion.

16. A method of detecting rupture of a cosmetic and reconstructive prosthesis comprising:
(a) surgically implanting a prosthesis containing a biologically compatible chemical indicator for indicating rupture of said prosthesis in a location of a patient body in need of said prosthesis; and
(b) detecting a change locally around said prosthesis for indication of leaking out of said indicator from said prosthesis.

17. The method of claim 16, wherein said change is a local skin color change.

18. The method of claim 16, wherein said change is a local sensation.

19. The method of claim 16, wherein said change is a local x-ray opacity change from that after said surgically implanting said prosthesis.
